(19) United States
(12) Patent Application Publication
(10) Pub. No.: US 2015/0369802 A1
(43) Pub. Date: Dec. 24, 2015

(54) Biomolecule Binding Composite Surfaces, Methods Of Making Such Surfaces, Devices Incorporating Such Surfaces, And Methods Of Using Such Surfaces In Biomolecule Binding Assays, And Devices Therefor

(71) Applicant: GVS North America, Inc., Sanford, ME (US)

(72) Inventors: Michael A Harvey, Spofford, NH (US); Debra W English, Rollingsford, NH (US); Yuander A Ju, Andover, MA (US); Emma R Smith, Dover, NH (US); Weibing Teng, Sanford, ME (US); Janet Yancey-Wrona, Falmouth, ME (US)

(21) Appl. No.: 14/545,782

(22) Filed: Jun. 18, 2015

Related U.S. Application Data
(60) Provisional application No. 61/998,124, filed on Jun. 18, 2014.

(51) Int. Cl. G01N 33/543

(52) U.S. Cl. CPC G01N 33/54366; G01N 33/54393

(57) ABSTRACT

The present invention relates to novel microporous, biomolecule binding composite surfaces. It also relates to processes for making such surfaces. It also relates to processes for incorporating such surfaces into assay devices, and the resulting assay devices. It also relates to readable biomolecule binding assay devices and methods of using such devices in protein microarrays or immunoassays.

### BACKGROUND OF THE INVENTION

(1) Field of the Invention

The present invention relates to novel biomolecule binding composite surfaces. It also relates to processes for making such surfaces. It also relates to processes for incorporating such surfaces into assay devices, and the resulting assay devices. It also relates to readable biomolecule binding assay devices and methods of using such devices in protein microarrays or immunoassays.

(2) Description of the Related Art, Including Information Disclosed Under 37 CFR 1.97 & 1.98

DNA and protein microarrays have become important methods to allow the simultaneous interrogation of multiple binding reactions (cf. Schena, M. et al., Science 270:467-469(1995), Duggan, D. J., et al., Nature Genetics 21:10-14, (1999), MacBeath, G. and Schrieber, S. L. Science 289:1760-1763(2000). Testing multiple samples at the same time for binding activity against all the elements of a given array significantly increases the power of parallel processing with minimal sample volumes.

Protein (antigen) microarrays have been recognized as being useful for vaccine development and the serodiagnosis of infectious diseases. (See Bacarese-Hamilton T et alia, Biotechniques 33:S24-S29 (December 2002).) Arrays with controls and calibrators have been shown on glass slides, but the art has recognized that “the development, of protein arrays for research and clinical applications has lagged behind, because of the poor stability of proteins, complex coupling chemistries, high variability, and weak detection signals. High density protein arrays remain difficult to generate and validate for clinical use”. (See Bacareseet alia at S26).

Porous membranes have been used as a reaction surface to support bio-molecular reactions for many years. The role of the membrane in these reactions is to provide a surface to facilitate separation of bound and free reactants. To accommodate this role, surfaces employed should be able to bind at least one of a binding pair in sufficient quantity to permit detection of binding to a second reactant. Surface that do not bind reactants nonspecifically are particularly advantageous.

In addition to providing a means to separate bound from free reactants the surface must be compatible with detection methods. Detection methods include isotopic detection, light interactive direct measurements such as absorption and fluorescence and visual detection. Many surfaces employed in binding reactions have high surface area to help maintain a locally high concentration of the primary binding reagent. This increases the sensitivity of the reaction and improves the speed of the reaction. However, high surface area materials have increased opportunity to bind reactants nonspecifically leading to background interferences. High surface area carriers can also interfere with light based measurements by absorbing or scattering light or by exhibiting autofluorescence.

Track etched polyester membranes or materials have been used as component in a biomolecule binding assay or assay device. For example, in U.S. Pat. No. 8,187,895 issued to Benjamin Feldman et alia discloses a small volume, in vitro analyte sensor and methods of making that sensor. Track etched membrane is used as a “porous cover” for the sensor. In the family of patents U.S. Pat. No. 8,101,431, U.S. Pat. No. 8,105,849, or U.S. Pat. No. 7,781,226, John McDivitt et alia disclose a point of care instrument for microarray analysis. Fluids and reagents are integrated into self-contained cartridges containing sensor elements. A membrane is used to filter the analytes of interest from a fluid stream. For example, if microbes represent the analyte of interest, then a track-etched polycarbonate membrane is used.

### BRIEF SUMMARY OF THE INVENTION

Methods and devices to provide and improve high throughput analysis of biomolecules (nucleic acids, proteins etc.) are important for elucidation of protein function, diagnostic testing, drug discovery, and drug target identification. One set of technologies that have improved the simultaneous interrogation of large numbers of biomolecules is the use of microarrays. Microarrays are ordered displays of molecules generally immobilized on a surface. Protein microarrays are tools used to measure protein concentration and function in biological samples. Their value is the simultaneous interrogation of small biological samples for a large number of different proteins, or biomarkers. The expression patterns that result from this type of analysis can be used to understand disease progression and response to therapy of individual patients. Ultimately, they may be used as tools for personalized medicine or as a companion diagnostic device.

Two important types of protein microarrays suitable for the present invention are antibody arrays and reverse phase protein arrays. Antibody arrays typically are research tools where multiple proteins are detected in a single sample. Antibody arrays provide a means to screen for multiple markers or diseases simultaneously. These arrays are most often sandwich immunoassays. Reverse phase protein arrays (RPPA) typically are a diagnostic tool where multiple patient samples can be queried simultaneously for biomarker expression. RPPA require the quantitative capture of all the proteins in tumor cell lysates followed by interrogation with multiple antibodies.

One of the most sensitive detection methods for both types of arrays is fluorescence. Fluorescent detection for protein microarrays and immunoassays has proven to be one of the most sensitive technologies, and instrumentation exists that is compatible with both array and multiwell plate formats. However, limitations of sensitivity can result from fluorescent background created by a combination of nonspecific binding events, optical interferences from the platform and low protein binding capacity.

An object of the invention is to provide an addressable fluorescent protein microarray with multiple surfaces that can bind many different proteins, maintains the protein three dimensional structure, immobilize them in sufficient quantity to allow for sensitive, rapid detection, and allow for surface-to-surface variability on each binding surface.

A second object of the invention is to be able to interrogate sensitively the same array of proteins with different samples or binding partners. Because specific protein binding partners are generally rare, any technique which allows the use of minimum quantities is preferred.

A third object of the invention is to use a convenient set of methodologies to allow high throughput techniques. In particular, an object of the invention is to use the “micro plate” 96 well format, which is based on 9 mm spacings of reaction areas which are 7 mm either in diameter or square. Many pipetting aids, detection instrumentation, liquid handling systems and robotics have been designed to conform to this format.

A fourth object of the present invention is to provide for parallel processing of substantially identical microarrays on multiple samples.

A fifth object of the present invention is to provide a microarray binding surface with significantly enhanced sensitivity, including less assay signal attenuation.

A sixth object of the present invention is to provide a microarray binding surface with significantly reduced background noise and enhanced sensitivity.

The high binding capacity, sensitivity and reproducibility of the present invention make them ideal for Reverse Phase Protein Arrays (RPPAs) used for biomarker discovery and characterization and in clinical trials. The present invention allows for quantitative binding across the broad dynamic range of protein concentration found in complex biological samples. The expression of specific proteins is detected with antibodies to the biomarkers of interest.

The high reproducibility and microporous, interstitially coated structure of the present invention is highly suitable for use in protein arrays used to diagnose infection and autoimmune diseases and for vaccine development and immunity monitoring. In protein arrays, a purified protein can be spotted on the biomolecule binding composite surface. The array can be used to detect the presence of antibodies or other binding proteins in clinical or experimental samples.

Biomolecule Composite Binding Surface

In a first group of embodiments, as seen in FIG. 1, the biomolecule composite binding surface is comprised of a track etched porous surface having an average pore size of about 40 um or less, a plurality of interstitial surfaces forming the pores of the track etched porous surface, and a microporous, biomolecule binding polymer dispersed about the non-interstitial surfaces and at least the interstitial surfaces of the track etched porous surface. (Throughout this disclosure, reference to the coating of the non-interstitial surfaces of a track etched porous surface means that at least some of such non-interstitial surfaces may be coated. It does not mean that the present invention requires any, much less all, of such non-interstitial surfaces to be coated.) The coated, porous surface is capable of achieving a dense loading of protein in a small area and can provide a high signal to noise ratio when used in light energy based assays. An advantage of the present invention is that one conduct quantitative multiplexed protein microimmunoassays on a microarray device.

Whereas in the prior art, porous nitrocellulose membranes were cast typically in thicknesses of about 100 um to 150 um if unsupported, or about 8 um to 14 um if supported by a transparent glass slide, the present invention allows unsupported castings on surfaces of about 1 um to 5 um.

Process for Making a Biomolecule Composite Binding Surface

In a second group of embodiments, the process for making the above described biomolecule composite binding surface comprises the following steps. First one track etches a defined length of a membrane surface selected from the group consisting of polyester polymers or polycarbonate polymers so as to form a track etched porous surface having a plurality of interstitial surfaces forming the pores of the track etched porous surface. Next, one forms a binding polymer solution comprised of a biomolecule binding polymer and a volatile polymer solvent. Third, one contacts the track etched porous surface with the binding polymer solution. Finally, one removes the polymer solvent from the contacted binding polymer solution so as to disperse the microporous, biomolecule binding polymer about the non-interstitial surfaces and at least the interstitial surfaces of the track etched porous surface.

Devices Using a Biomolecule Composite Binding Surface

In a third group of embodiments, devices using the above described biomolecule composite binding surface comprise two combined elements. The first element is a support member having a support surface. The second element is a composite membrane attached to the support surface, the composite membrane being comprised of a track etched porous surface having an average pore size of less about 40 um or less, a plurality of interstitial surfaces forming the pores of the track etched porous surface, and a microporous, biomolecule binding polymer dispersed about the non-interstitial surfaces and at least the interstitial surfaces of the track etched porous surface.

Process for Making a Device Using a Biomolecule Composite Binding Surface

In a fourth group of embodiments, processes for making a microporous, biomolecule binding device using the above described biomolecule composite binding surface comprise the following steps. First, one selects a support member. Next, one places a dry film adhesive onto the support member. Third, one places on the dry film, heat-activated adhesive a composite membrane comprised of a track etched porous surface having an average pore size of about 40 um or less, a plurality of interstitial surfaces forming the pores of the track etched porous surface, and a microporous, biomolecule binding polymer dispersed about the non-interstitial surfaces and at least the interstitial surfaces of the track etched porous surface. Finally, one applies thermal energy to the adhesive sufficient to cause the adhesive to adhere the composite membrane to the support member.

Process for Using a Readable Biomolecule Binding Assay Devices Having a Composite Binding Surface

In a fifth group of embodiments, processes for using the biomolecule binding device in immunoassay and a protein microarray comprise the following steps. A process for detecting an analyte signal from a biomolecule binding assay having a assay signal generation reagent comprises, first, selecting a biomolecule binding device comprised of a support member having a support surface; and a composite membrane attached to the support surface, the composite membrane being comprised of a track etched porous surface having an average pore size of about 40 um or less, a plurality of interstitial surfaces forming the pores of the track etched porous surface, and a microporous, biomolecule binding polymer dispersed about the non-interstitial surfaces and at least the interstitial surfaces of the track etched porous surface. Next, one places an analyte sample on the composite membrane, thereby allowing any biomolecule analyte in the sample to bind to the composite membrane. Third, the assay signal generation reagent is reacted with the bound biomolecule so as to form a labelled and bound biomolecule. Finally, one detects the labelled and bound biomolecule.

Readable Biomolecule Binding Assay Devices Having a Composite Binding Surface

In a sixth group of preferred embodiments, a readable biomolecule binding assay device for detecting an analyte signal from a biomolecule binding assay having a assay signal generation reagent device ready to be read, comprises the following elements. First, a biomolecule binding device comprises a support member having a support surface; and a composite membrane attached to the support surface, the composite membrane being comprised of a track etched porous surface having an average pore size of about 40 um or less, a plurality of interstitial surfaces forming the pores of the track etched porous surface, and a microporous, biomolecule binding polymer dispersed about the non-interstitial surfaces and at least the interstitial surfaces of the track etched porous surface. Next, an analyte sample is disposed on the composite membrane, thereby allowing any biomolecule analyte in the sample to be bound to the composite membrane. Finally, the assay signal generation reagent is present, having been reacted with any bound biomolecule so as to form a labelled and bound biomolecule.

For the purposes of the present invention, “microporous” refers to a pore size ranging from about 40 um down to about 0.5 um.

### BRIEF DESCRIPTION OF THE DRAWINGS

FIG. 1 is a cross sectional scanning electron microscope image of the present invention showing the composite binding surface, including the interstitial surfaces of the track etched membrane having a coating of microporous binding polymer about those surfaces.

FIG. 2 is a cross sectional schematic view of a preferred dry casting process embodiment of the present invention using a micro-gravure coating device.

FIG. 3 is a cross sectional schematic view of a preferred wet casting process embodiment of the present invention using a micro-gravure coating device.

FIG. 4 is a graph showing a log/log plot of the total chemiluminescent signal versus protein binding for a transferrin immunodot assay, comparing an example of the prior art to one of the present invention.

FIG. 5 is a graph showing the signal to noise ratio versus protein binding for a transferrin immunodot assay, comparing an example of the prior art to one of the present invention.

FIG. 6 is a graph showing the relative signal versus a series of protein serum dilutions for a transferrin immunodot assay, comparing an example of the prior art to one of the present invention.

### DETAILED DESCRIPTION OF THE INVENTION

Preferred Biomolecule Composite Binding Surfaces

In a first group of preferred embodiments, the biomolecule composite binding surface is comprised of a track etched porous surface having an average pore size of about 20 um or less, a plurality of interstitial surfaces forming the pores of the track etched porous surface, and a microporous, biomolecule binding polymer dispersed about the non-interstitial surfaces and the interstitial surfaces of the track etched porous surface. The surface is capable of achieving a dense loading of protein in a small area and can provide a high signal to noise ratio when used in light energy based assays. An advantage of the present invention is that one conduct quantitative multiplexed protein microimmunoassays on a microarray device.

Preferably, the microporous, biomolecule binding polymer is dispersed about substantially all of the interstitial surfaces. In addition, the microporous, biomolecule binding polymer has an average thickness about the interstitial surfaces of at least 1 um and/or an average thickness about the interstitial surfaces of at least 3 um.

Suitable materials for the microporous, biomolecule binding polymer include nitrocellulose, polyvinylidene fluoride, polymers activated with a leaving group such as sulfonyl chloride activated cellulose, or nylon. A most preferred material is nitrocellulose.

Preferably, the track etched porous surface is no more than about 15 um thick. In addition, the track etched porous surface has an average pore size of about 20 um or less and/or a pore density of between about 10⁴ to 10⁹ per cm².

Suitable materials for tracked etched porous surfaces include polyester polymers, polyimide polymers, fluorinated polymers (such as polyvinylidene fluoride), polyethylene naphthalate polymers, polypropylene polymers, or polycarbonate polymers. A most preferred material is polyester polymers (PET).

The present invention is suitable for preparing continuous or bulk lengths of composite membrane that can be further processed for incorporation into assay devices. Thus, preferably, the track etched porous surface of the present invention may have a length of at least one meter.

Preferred Processes for Making a Biomolecule Composite Binding Surface

In a second group of preferred embodiments, the process for making the above described biomolecule composite binding surface comprises the following steps. First one track etches a defined length of a membrane surface selected from the group consisting of polyester polymers or polycarbonate polymers so as to form a track etched porous surface having a plurality of interstitial surfaces forming the pores of the track etched porous surface.

Next, one forms a binding polymer solution comprised of a biomolecule binding polymer and a volatile polymer solvent. The binding polymer solution comprises a biomolecule binding polymer and a solvent capable of solubilizing the biomolecule binding polymer. Suitable biomolecule binding polymers include nitrocellulose, polyvinylidene flouride, polymers activated with a leaving group such as sulfonyl chloride activated cellulose, or nylon. Preferably, the binding polymer solution can have a total solids content of at least five percent dissolved total solids and/or a viscosity of at least two seconds, using the falling ball method, which is well known to those of ordinary skill in the art.

For example, in the case of nitrocellulose, one can use a number of solvent mixes as are known to those of ordinary skill in the art. For example, a solvent for nitrocellulose include includes ketones (such as acetone, methyl ethyl ketone), esters (such as ethyl acetate or butyl acetate) or glycol ethers (such as methyl glycol ether or ethyl glycol ether).

One can vary the total solids of the nitrocellulose from 5% to 10% of the volume of the binding polymer solution. In addition, one can add solution modifiers such as surfactants or thickeners such as glycerol. Preferably, solvent solution can vary in viscosity from two seconds to six seconds, using the falling ball method, which is well known to those of ordinary skill in the art.

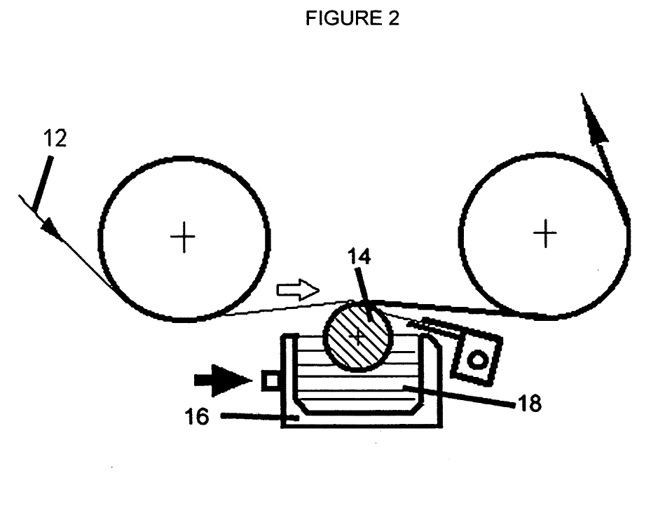

Third, as shown in FIG. 2, the track etched porous membrane surface (12) is contacted with a film of binding polymer solution, the film having a thickness of at least equal to the thickness of the track etched porous surface. Such a film can be made by the rotational movement of a roller (14) which is in partial contact with a reservoir (16) filled with the binding polymer solution (18). The defined length of the track etched porous surface can be moved along an axis transverse to a longitudinal non-rotational axis of the roller. The rotational movement (22) of the roller can oppose the movement of the track etched porous surface (24). The speed of the roller movement and the track etched porous surface movement should be adjusted to provide the desired thickness of the interstitial biomolecule binding surface. For example, while moving the track etched porous surface at a speed of about one meter per minute, the roller movement can vary between one half meter per minute and two meters per minute. Generally, the higher the ratio of binding polymer solution roller movement to track etched membrane movement, the thicker will be the coating of microporous, binding polymer about the interstitial surfaces of the track etched porous membrane. Moreover, use of the roller will coat the non-interstitial surfaces of at least one side of the track etched porous surface.

One can use a micro-gravure device to achieve these results. A number of manufacturers make such devices, which are commercially available. By using such a device, one can make rolls of finished microporous, biomolecule composite binding surface exceeding a meter in length, typically making a roll of at least 30 meters in length, but the process can easily be scaled up to make composite surface rolls of over 1000 meters in length.

Typically, the track etched porous membrane surface can be less than about 25 urn thick. The surface can have an average pore size of about 20 um or less. It can also have a pore density of between about 10⁴ to about 10⁹ per cm².

Finally, one removes the polymer solvent from the contacted binding polymer solution so as to disperse the biomolecule binding polymer about the non-interstitial surfaces and the interstitial surfaces of the track etched porous surface, creating the biomolecule composite binding surface. In the case of a volatile solvent, one can air dry to remove the polymer solvent. Conventional hot air blow drying can be used in the present processes to accelerate evaporation of a volatile solvent. Another option is to dry alone or in combination with a conventional heating oven. Typically, the upper drying temperature limit is from about 100 degrees C. to about 200 degrees C. As known to those of ordinary skill in the art, the optimal drying parameters depend upon the speed of draw of the composite surface and the solvent formulation used.

Preferably, these present processes result in the microporous, biomolecule binding polymer having an average thickness about the interstitial surfaces of at least 3 um, more preferably of at least 1 um.

A biomolecule composite binding surface (BCBS) made using the above processes was made and compared to a prior art microarray assay surface a commercially available FAST brand 16 pad slide made by GVS North America of Sanford, Me. USA. The FAST slide comprises nitrocellulose cast on a glass slide. The preferred BCBS surface was nitrocellulose bound to a polyester track etched membrane. The average thickness of the nitrocellulose on the BCBS surface was about 3 um, as determined by scanning electron microscope imaging. One mg/ml anti-IL-6 antibody solution was piezo printed onto the FAST slide and the BCBS surface using a piezo electric arrayer. IL-6 (0.5 ng/ml) was used as a source of to determine a signal to noise ratio. A conventional titration of IL-6 antigen was used to generate a standard curve for a conventional fluorescent immunoassay.

Both the FAST slide and the BCBS surface had a similar spot diameter for the analyte, about 180 um for the FAST slide and about 160 um for the BCBC surface. However, the signal to noise ratio of the IL-6 antigen fluorescent immunoassay was about 25 for the FAST slide compared to up to about 75 for the BCBS surface. The BCBS surface had a much more optimal signal to noise ratio while maintaining the overall assay sensitivity by having a similar spot size.

Preferred Devices Using a Biomolecule Composite Binding Surface

In a third group of preferred embodiments, devices using the above described biomolecule composite binding surface comprise a number of combined elements. The first element is a support member (typically transparent, such as a glass or a plastic polymer member) having a support surface. The second element is a composite membrane attached to the support surface, the composite membrane being comprised of a track etched porous surface having an average pore size of about 20 um or less, a plurality of interstitial surfaces forming the pores of the track etched porous surface, and a microporous, biomolecule binding polymer dispersed about the non-interstitial surfaces and the interstitial surfaces of the track etched porous surface. A third element is an adhesive placed between the support member and the composite membrane. The support member can have a perimeter area with the adhesive and the composite membrane being placed on the support member within a placement area defined by the perimeter area. A pad barrier can be connected to the support member about the perimeter area.

The present invention is highly suitable for use in a multiwell device. Typically, a multiwell biomolecule binding device comprises a multiwell plate having a well bottom support surface and a plurality of well, each such well having a well opening and being separate and distinct from the other wells. A plurality of well membranes is located, each well membrane being located in a separate well on the well bottom support surface. Each well membrane is comprised of a track etched porous surface having an average pore size of about 20 um or less, a plurality of interstitial surfaces forming the pores of the track etched porous surface. A microporous, biomolecule binding polymer is dispersed about the non-interstitial surfaces and the interstitial surfaces of the track etched porous surface; each well having a single well membrane placed therein. Preferably, an adhesive is located on at least a portion of the well bottom support in each well before the plurality of well membranes, between the well bottom surface and the individual well membrane. By such a combination of elements, each well membrane is adhered to its respective individual well bottom support surface.

The present invention has another advantage of not requiring a solid support surface. The biomolecule binding composite surface can be incorporated into a “through filtration” device architecture. For example, one can allow for the deposition of cell lysates in a vacuum application. Existing RPPA platforms (such as the FAST brand slides) do not permit this application.

Preferred Processes for Making a Device Using a Biomolecule Composite Binding Surface

In a fourth group of preferred embodiments, processes for making a biomolecule binding device using the above described biomolecule composite binding surface comprise the following steps. First, one selects a support member (typically transparent, such as a glass or a plastic polymer member). The support member has a perimeter area. Next, one places a dry film adhesive onto the support member. Third, one places on the dry film, heat-activated adhesive a composite membrane comprised of a track etched porous surface having an average pore size of about 20 um or less, a plurality of interstitial surfaces forming the pores of the track etched porous surface, and a microporous, biomolecule binding polymer dispersed about the non-interstitial surfaces and the interstitial surfaces of the track etched porous surface. The adhesive and the composite membrane are placed on the support member within a placement area defined by the perimeter area. A pad barrier is also connected to the support about the perimeter area.

In the last step, one applies thermal energy to the adhesive sufficient to cause the adhesive to adhere the composite membrane to the support member.

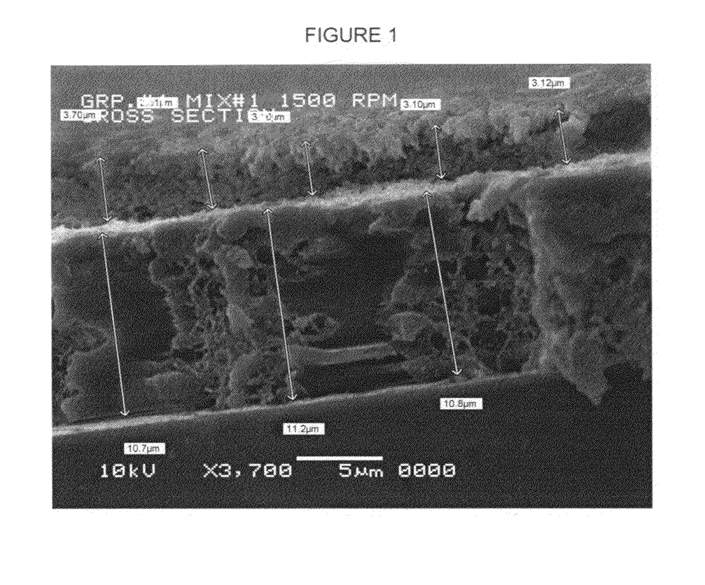

The present invention also is highly suitable for novel processes to make a multiwell device. Typically, the process for making a multiwell biomolecule binding device made by the novel processes comprises first selecting a multiwell plate having a well bottom support surface and a plurality of well. Each such well has a well opening and is separate and distinct from the other wells. Next, one locates over the well openings a composite membrane comprised of a track etched porous surface having an average pore size of about 20 um or less, a plurality of interstitial surfaces forming the pores of the track etched porous surface, and a microporous, biomolecule binding polymer dispersed about In a first group of embodiments, as seen in FIG. 1, the biomolecule composite binding surface is comprised of a track etched porous surface having an average pore size of about 40 um or less, a plurality of interstitial surfaces forming the pores of the track etched porous surface, and a microporous, biomolecule binding polymer dispersed about the non-interstitial surfaces and the interstitial surfaces of the track etched porous surface. (Throughout this disclosure, reference to the coating of the non-interstitial surfaces of a track etched porous surface means that at least some of such non-interstitial surfaces may be coated. It does not mean that the present invention requires any, much less all, of such non-interstitial surfaces to be coated.). Finally, one cuts or otherwise separates the composite membrane into a plurality of well membranes that can be placed or otherwise dropped about the well bottom support surface, each separate well membrane being located in a separate well on the well bottom support surface. One way to simultaneously separate the well membranes is to apply a cutting die onto the composite membrane and pushing the cutting die into and through the composite membrane, forming the plurality of separate well membranes.

In one preferable process, one can locate an adhesive on at least a portion of the well bottom support in each well before the plurality of well membranes is formed. Thus, an adhesive would be placed between the well bottom surface and each individual well membrane. By such a process for combining elements, each well membrane is adhered to its respective individual well bottom support surface.

Preferred Processes for Using a Readable Biomolecule Binding Assay Devices Having a Composite Binding Surface

In a fifth group of preferred embodiments, novel processes for using the biomolecule binding device in readable biomolecule binding assay comprise the following steps. First, one selects a biomolecule binding device comprised of a support member having a support surface; and a composite membrane attached to the support surface. The composite membrane is comprised of a track etched porous surface having an average pore size of about 20 um or less, a plurality of interstitial surfaces forming the pores of the track etched porous surface, and a microporous, biomolecule binding polymer dispersed about the non-interstitial surfaces and the interstitial surfaces of the porous surface. Next, one places an analyte sample on the composite membrane, thereby allowing any biomolecule analyte in the sample to bind to the composite membrane. Third, one reacts the assay signal generation reagent with the bound biomolecule so as to form a labelled and bound biomolecule. Finally, one detects the labelled and bound biomolecule.

The above described architecture can be used to generate highly sensitive assays with a large signal to noise ratio in a variety of formats, including devices immunoassays, protein microarrays, nucleic acid assays, receptor binding assays, DNA protein binding reactions, or immobilized enzymatic based reactions.

Preferred Readable Biomolecule Binding Assay Devices Having a Composite Binding Surface

In a sixth group of preferred embodiments, a readable biomolecule binding assay device for detecting an analyte signal from a biomolecule binding assay having a assay signal generation reagent device ready to be read, comprises the following elements. First, a biomolecule binding device comprises a support member having a support surface; and a composite membrane attached to the support surface, the composite membrane being comprised of a track etched porous surface having an average pore size of about 20 um or less, a plurality of interstitial surfaces forming the pores of the track etched porous surface, and a microporous, biomolecule binding polymer dispersed about about the non-interstitial surfaces and the interstitial surfaces of the porous surface. Next, an analyte sample is disposed on the composite membrane, thereby allowing any biomolecule analyte in the sample to be bound to the composite membrane. Finally, the readable assay signal generation reagent is present, having been reacted with any bound biomolecule so as to form a labelled and bound biomolecule.

### Enhanced Sensitivity of Analyte Detection

The present invention provides unexpected benefits in the performance characteristics of readable biomolecule binding assay devices. Normally, one of ordinary skill in the art would expect that a lower surface area protein binding capacity for a selected reaction surface for a biomolecule binding assay would result in the assay having a lower sensitivity. However, such is not the case with the present invention. Despite having a lower surface area protein binding capacity that known conventional surface (such as supported nitrocellulose), the present invention has a higher sensitivity.

The protein binding capacity of the present invention was determined by subjecting separately 1 cm discs of the present (made in accordance with methods as described above) and 1 cm discs of supported nitrocellulose to 5 mg/ml BSA in PBS overnight. Following extensive washings with PBS to remove unbound protein, the discs were assayed for bound protein using a BioRad brand BCA protein assay.

As can be seen in Table 1 below, the thin interstitial layer (3 um) of nitrocellulose on the BCBS binds less total protein than the same surface area on a conventional supported nitrocellulose membrane.

TABLE 1
Surface Area Binding of BSA to BCBS
versus supported nitrocellulose

| Membrane | BSA/cm2 |
|---|---|
| BCBS | 6.23 |
| Supported Nitrocellulose | 49.0 |

However, as can been in Table 2 below, the density of protein binding is nearly 5 times greater on BCBS as opposed to supported nitrocellulose membrane. This result is manifest in the enhanced signal/noise ratios that can be observed in in an immunodot assay shown below.

TABLE 2
Total Binding of BSA to BCBS versus supported nitrocellulose

| Membrane | ug BSA/cm3 |
|---|---|
| BCBS | 17793 |
| Supported Nitrocellulose | 3769 |

Enhanced density protein binding results in an assay with an enhanced signal to noise ratio. To show this benefit, a conventional transferrin immunodot assay was performed on both the present invention and supported 0.2 micron porosity nitrocellulose membrane. Declining concentrations of human transferrin were applied separately to a BCBS surface and to a supported nitrocellulose membrane by filtration in a multiwell dot blot apparatus made by Schleicher & Schuell, Inc.

Following conventional immunodot assay procedures, each membrane was blocked and each membrane was incubated with a 1 to 20,000 dilution of rabbit anti-human transferrin. The membranes were then washed and reacted with a 1 to 25,000 dilution of horseradishperoxidase conjugated secondary antibody.

Chemiluminescence signal was generated using a Thermo Scientific's SuperSignal West Dura Extended Duration Substrate luminol component. A working solution comprised of a 1 to 1 chemiluminescent substrate solution of stable peroxide solution and the luminol/enhancer solution was made. The amount of chemiluminescent substrate solution was about 0.1 ml per cm2 of membrane. Each membrane was incubated for 5 minutes with the working solution. After placing the membrane into a plastic sheet, any excess liquid and air bubbles were removed. Chemiluminescent signal was captured and analyzed using a UVP Imaging System made by UVP, LLC of Upland, Calif.

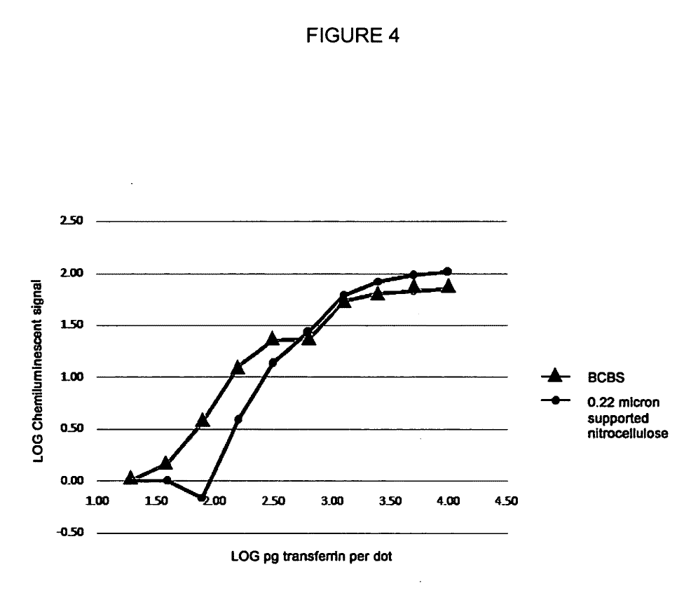

As can be seen from the plotted results in FIG. 4, the above-described comparative dot blot immunoassays demonstrate that the present invention has enhanced sensitivity of detection at low concentrations of transferrin even though the binding capacity is less than the conventional membrane. Surprisingly, though the present invention has a lower amount of bound protein (antibody) on a defined planar surface area, it can bind a greater amount of within the volume defined by an assay surface area.

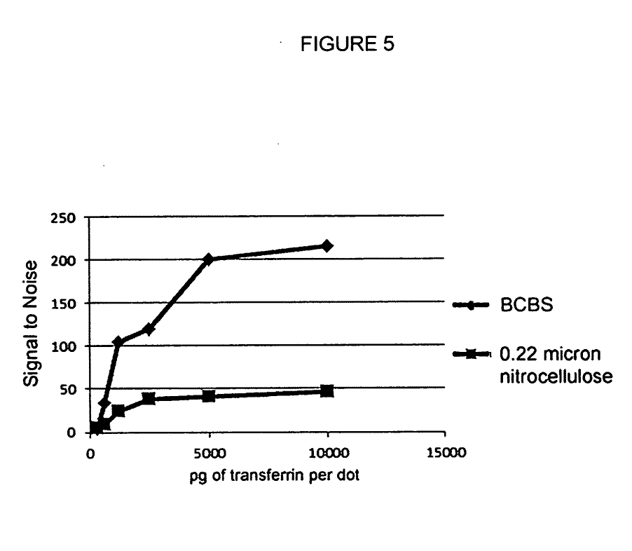

As can be seen from the plotted results in FIG. 5, the above-described comparative dot blot immunoassays demonstrate that the present invention also supports immunoassays with enhanced sensitivity when compared to traditional microporous membranes. Surprisingly, the present invention can achieve more sensitive detection than the conventional surface. The sensitivity of the enhanced protein binding, greater than on the traditional membrane, is reflected in the higher signal to noise (S/N) ratios achieved by the present invention. Such higher S/N ratios are particularly observed at lower antigen concentrations.

Human serum, containing transferrin was electrophoresed on 12% SDS-PAGE gels and then electrophoretically, transferred onto either BCBS or a standard 0.2 um supported NC membrane using Life Technologies XCell SureLock Mini-Cell/blot module. Serum dilution started at 1:175 followed by three, 1:2 serial dilutions. Transferrin was detected using a rabbit anti-human transferrin primary antibody and goat anti-rabbit IgG-HRP conjugate

After the transfer, the membrane was rinsed for 5 minutes in 1× Tris buffered saline (TBS). Following the TBS rinse, the membrane was blocked for 1 hour in TBS with 0.5% nonfat dry milk with rocking. All incubation steps occurred at room temperature. After blocking, the membrane was washed 3 times with TBS, 0.05% Tween 20 (TBS/Tw20). Each wash was 5 minutes. The membrane was incubated with the primary antibody rabbit anti human transferrin (1:1000 to 1:3000; ˜3 to 8.5 μg/ml) which was diluted in TBS/Tw20 for 1 hour with rocking.

Once the antibody solution was discarded and the membrane washed 3 times as described above, the membrane was incubated for 1 hour in horseradish peroxidase conjugated secondary antibody diluted 1:20,000 in TBS/Tw20. The HRP-conjugate was decanted and the membrane washed 5 times to remove unbound HRP-conjugate.

As with the immunodot assay, chemiluminescence was generated using a Thermo Scientific's SuperSignal West Dura Extended Duration Substrate luminol component. A working solution comprised of a 1 to 1 chemiluminescent substrate solution of stable peroxide solution and the luminol/enhancer solution was made. The amount of chemiluminescent substrate solution was about 0.1 ml per cm2 of membrane. Each membrane was incubated for 5 minutes with the working solution. After placing the membrane into a plastic sheet, any excess liquid and air bubbles were removed. Chemiluminescent signal was captured and analyzed using a UVP Imaging System made by UVP, LLC of Upland, Calif.

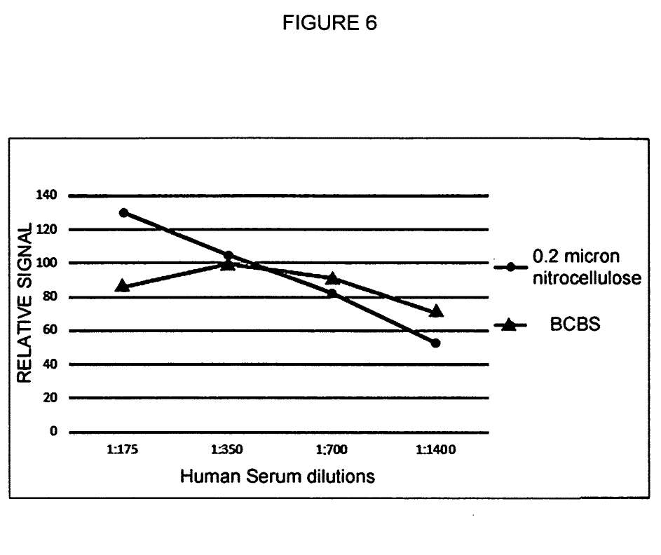

The results can be seen in FIG. 6. It shows the plotted signal from the two Western blots performed on the compared membrane surfaces. At higher dilutions, the present invention has a higher signal than the prior art, demonstrating the enhanced sensitivity for detection of an analyte in a biomolecule binding assay by using the present invention.

### Alternative Preferred Processes for Making Biomolecule Composite Binding Surfaces

An alternative to the dry casting processes for making biomolecule binding composite surfaces embodiments described above is a wet casting series of embodiments. Wet casting embodiments of the present processes for making the above described biomolecule composite binding surfaces comprise the following steps.

First one track etches a defined length of a membrane surface, preferably selected from the group consisting of polyester polymers or polycarbonate polymers, so as to form a track etched porous surface having a plurality of interstitial surfaces forming the pores of the track etched porous surface.

Next, one forms a binding polymer solution comprised of a biomolecule binding polymer and a volatile polymer solvent. The binding polymer solution comprises a biomolecule binding polymer and a solvent capable of solubilizing the biomolecule binding polymer. Suitable biomolecule binding polymers for wet casting include polyvinylidene flouride (PVDF) or nylon. Preferably, the binding polymer solution can have a total solids content of at least five percent dissolved total solids and/or a viscosity of at least two seconds, using the falling ball method, which is well known to those of ordinary skill in the art.

For example, in the case of PVDF, one can use a number of solvent mixes as are known to those of ordinary skill in the art. For example, a solvent for PVDF include includes a lactam compound, such as N-Methyl-2-pyrrolidone (NMP).

One can vary the total solids of the PVDF from 5% to 10% of the volume of the binding polymer solution. In addition, one can add solution modifiers such as hydrochloric acid (HCL). Preferably, solvent solution can vary in viscosity from two to six seconds, using the falling ball method, which is well known to those of ordinary skill in the art.

Third, one contacts the track etched porous surface with a film of binding polymer solution, the film having a thickness of at least equal to the thickness of the track etched porous surface. Such a film can be made by the rotational movement of a roller which is in partial contact with a reservoir filled with the binding polymer solution. The defined length of the track etched porous surface can be moved along an axis transverse to a longitudinal non-rotational axis of the roller. The rotational movement of the roller can oppose the movement of the track etched porous surface. The speed of the roller movement and the track etched porous surface movement should be adjusted to provide the desired thickness of the interstitial microporous, biomolecule binding surface. For example, while moving the track etched porous surface at a speed of about one meter per minute, the roller movement can vary between one half meter per minute and two meters per minute. Generally, the higher the ratio of binding polymer solution roller movement to porous surface movement, the thicker will be the coating of microporous, binding polymer about the interstitial surfaces of the track etched porous membrane.

One can use a micro-gravure device to achieve these results. A number of manufacturers make such devices, which are commercially available. By using such a device, one can make rolls of finished biomolecule composite binding surface exceeding a meter in length, typically making a roll of at least 30 meters in length, but the process can easily be scaled up to make composite surface rolls of over 1000 meters in length.

Typically, the track etched porous surface can be less than about 15 um thick. The surface can have an average pore size of about 40 um or less, preferably about 20 um or less. It can also have a pore density of between about 10⁴ to about 10⁹ per cm².

Finally, one removes the polymer solvent from the contacted binding polymer solution so as to disperse the microporous, biomolecule binding polymer about the non-interstitial surfaces and the interstitial surfaces of the track etched porous surface, creating the biomolecule composite binding surface.

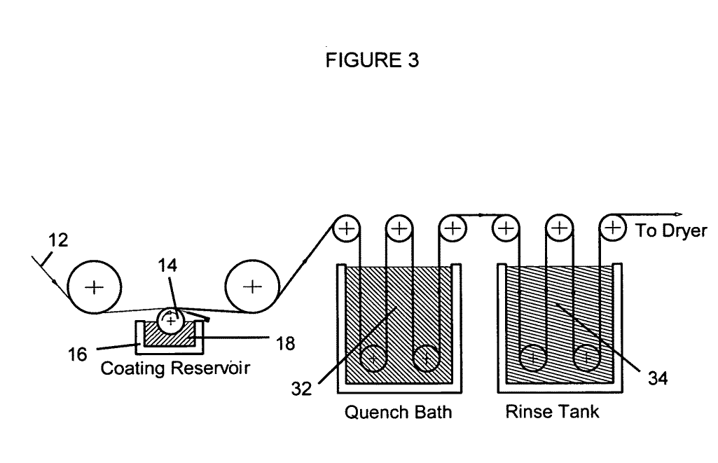

With wet casting embodiments as shown in FIG. 3, this step begins with placing the track etch porous surface (12) containing the interstitially cast biomolecule binding polymer into a phase inversion solution or bath (32). As is known to those of ordinary skill in the art, typically a phase inversion bath (also known as a quenching bath or solution) comprises a mixture of the binding polymer solvent and another liquid non-solvent. For example, in the case of PVDF, one can use a binding polymer solution comprised of about 83.5% NMP, about 13.5% PVDF, and about 300 mL HCl and a phase inversion solution comprised of about 50 NMP and about 50% water. The composite membrane should be in contact with such a solution, typically traveling through the bath as shown in FIG. 3, long enough for the binding polymer undergo phase inversion, forming a microporous structure about the filaments and structure of the track etched porous membrane.

After being placed in the quenching bath, the composite membrane is placed into a rinsing bath (34). The rinse bath solution removes any remaining dissolved binding polymer solids not bound to the track etched porous membrane, along with any solvent or non-solvent solutions. In the case of PVDF, a rinse solution may be comprised of water.

Finally, with wet casting preferred embodiments of the present processes, one can air dry the biomolecule composite binding surface so as to remove any rinse solution. Conventional hot air blow drying can be used in the present processes to accelerate evaporation of the rinse solution. Another option is to dry alone or in combination with a conventional heating oven. Typically, the upper drying temperature limit is from about 100 degrees C. to about 200 degrees C. As known to those of ordinary skill in the art, the optimal drying parameters will depends upon the speed of draw of the composite surface and the rinse solution used.

Preferably, these wet casting processes result in the biomolecule binding polymer having an average thickness about the interstitial surfaces of at least 3 um, more preferably of at least 1 um.

Preferred Pretreated Biomolecule Composite Binding Surfaces

Another alternative set of preferred embodiments of biomolecule composite binding surfaces in the present invention comprise surfaces in which the track etched membrane has received a surface pre-treatment that enhances either the binding capacity or the binding attraction of the binding polymer to the track etched membrane. For example, in the case of polyester polymer based track etched membrane, one can enhance the binding capacity of a nitrocellulose binding polymer (as part of a binding polymer solution used in the present processes) by pretreating the polyester polymer track etched membrane with a silane.

As is known to those of ordinary skill in the art, one can prepare a diluted silane composition comprised of about 1% (3-aminopropyl)triethoxysilane (APTES) silane in an ethanol solvent. The track etched membrane is placed in a coating bath or solution of the about 1% silane solution for a time sufficient to allow reaction of the silane with the polyester polymer. In a continuous, or roll driven system, the track etched membrane can travel through such a solution for about 15 seconds at a speed of about one meter per minute. Finally, one dries the silane coated track etched membrane using conventional means.

The present invention is highly suitable for receiving printed reagents such as protein arraying spots arranged using conventional printing means in regular patterns with a uniform centered spacing. As known to those of ordinary skill in the art, one typically uses solutions of about 0.5 mg/ml of protein. Each spot can have a diameter of between about 50 um and about 1000 um. Calibrator spots can be in series of five variable known concentrations.

Kits can be made that incorporate the above devices along with any combination of associated equipment or reagents including slide holders, slide chambers, fluorescent binding reagents, or informatic software for generating standard curves and interpretative reading results of the microarray on the device.

The present invention can be used to detect the presence of autoreactive antibodies in a patient having an autoimmune disease, antibodies to viral diseases, antibodies to bacterial diseases, or antibodies to allergic reactions. The present invention can be used for multiplexed infectious disease testing against hepatitis A, B, or C, Epstein-Barr virus, human papilloma virus, Lyme Disease and others.

All publications or unpublished patent applications mentioned herein are hereby incorporated by reference thereto.

Other embodiments of the present invention are not presented here which are obvious to those of ordinary skill in the art, now or during the term of any patent issuing from this patent specification, and thus, are within the spirit and scope of the present invention.

We claim the following:

1. A composite membrane comprised of a track etched porous surface having an average pore size of about 40 um or less, a plurality of interstitial surfaces forming the pores of the track etched porous surface, and a microporous, biomolecule binding polymer dispersed about at least the interstitial surfaces.

2. The composite membrane of claim 1 wherein the microporous, biomolecule binding polymer is dispersed about substantially all of the interstitial surfaces.

3. The composite membrane of claim 1 wherein the track etched porous surface is selected from the group consisting of polyimide polymers, fluorinated polymers, polyethylene naphthalate polymers, polypropylene polymers, polycarbonate polymers, polyimide polymers, polyester polymers, or polycarbonate polymers.

4. The composite membrane of claim 3 wherein the track etched porous surface is a polyester polymer.

5. The composite membrane of claim 1 wherein the microporous, biomolecule binding polymer is selected from the group consisting of nitrocellulose, polyvinylidene flouride, or nylon.

6. The composite membrane of claim 5 wherein the microporous, biomolecule binding polymer is nitrocellulose.

7. The composite membrane of claim 1 wherein the track etched porous surface is no more than about 15 um thick.

8. The composite membrane of claim 1 wherein the track etched porous surface has an average pore size of about 20 um or less.

9. The composite membrane of claim 1 wherein the track etched porous surface has a pore density of between about 10⁴ to about 10⁹ per cm²

10. The composite membrane of claim 1 wherein the microporous, biomolecule binding polymer has an average thickness about the interstitial surfaces of at least 1 um.

11. The composite membrane of claim 1 wherein the microporous, biomolecule binding polymer has an average thickness about the interstitial surfaces of at least 3 um.

12. The composite membrane of claim 1 wherein the track etched porous surface has a length of at least one meter.

13. A process for making a composite membrane comprised of a track etched porous surface having an average pore size of about 40 um or less, a plurality of interstitial surfaces forming the pores of the track etched porous surface, and a microporous, biomolecule binding polymer dispersed about at least the interstitial surfaces comprising;
a) track etching a defined length of a membrane surface so as to form a track etched porous surface having a plurality of interstitial surfaces forming the pores of the track etched porous surface;
b) forming a binding polymer solution comprised of a microporous, biomolecule binding polymer and a volatile polymer solvent;
c) contacting the track etched porous surface with the binding polymer solution; and
d) removing the polymer solvent from the contacted binding polymer solution so as to disperse the microporous, biomolecule binding polymer about at least the interstitial surfaces of the track etched porous surface.

14. The process of claim 13 wherein the track etched porous surface is selected from the group consisting of polyimide polymer's, fluorinated polymers, polyethylene naphthalate polymers, polypropylene polymers, polycarbonate polymers, polyester polymers or polycarbonate polymers.

15. The process of claim 13 wherein the track etched porous surface is contacted with a film of the binding polymer solution, the film having a thickness of at least equal to the thickness of the track etched porous surface.

16. The process of claim 15 wherein the film is made by the rotational movement of a roller which is in partial contact with a reservoir filled with the binding polymer solution.

17. The process of claim 16 wherein the defined length of the track etched porous surface is moved along an axis transverse to a longitudinal non-rotational axis of the roller.

18. The process of claim 17 wherein the rotational movement of the roller opposes the movement of the track etched porous surface.

19. The process of claim 18 wherein the microporous, binding polymer solution comprises a microporous, biomolecule binding polymer and a solvent capable of solubilizing the microporous, biomolecule binding polymer.

20. The process of claim 18 wherein the microporous, binding polymer solution has a total solids content of at least five percent dissolved total solids

21. The process of claim 18 wherein the microporous, binding polymer solution has a viscosity of at least two seconds.

22. The process of claim 13 wherein the microporous, biomolecule binding polymer is selected from the group consisting of nitrocellulose, polymers activated with a leaving group such as sulfonyl chloride activated cellulose, polyvinylidene flouride, or nylon.

23. The process of claim 16 wherein the microporous, biomolecule binding polymer is nitrocellulose.

24. The process of claim 13 wherein the track etched porous surface is no more than about 15 um thick.

25. The process of claim 13 wherein the track etched porous surface has an average pore size of about 20 um or less.

26. The process of claim 13 wherein the track etched porous surface has a pore density of between about 10⁴ to about 10⁹ per cm².

27. The process of claim 13 wherein the microporous, biomolecule binding polymer has an average thickness about the interstitial surfaces of at least 1 um.

28. The process of claim 13 wherein the microporous, biomolecule binding polymer has an average thickness about the interstitial surfaces of at least 3 um.

29. The process of claim 13 wherein the track etched porous surface has a length of at least one meter.

30. A biomolecule binding device comprising:
a) a support member having a support surface; and
b) a composite membrane attached to the support surface, the composite membrane being comprised of a track etched porous surface having an average pore size of about 40 um or less, a plurality of interstitial surfaces forming the pores of the track etched porous surface, and a microporous, biomolecule binding polymer dispersed about at least the interstitial surfaces.

31. The biomolecule binding device of claim 30 wherein the track etched porous surface is selected from the group consisting of polyimide polymers, fluorinated polymers, polyethylene naphthalate polymers, polypropylene polymers, polycarbonate polymers, polyester polymers, or polycarbonate polymers.

32. The biomolecule binding device of claim 30 also comprising and an adhesive located between the support member and the composite membrane.

33. The biomolecule binding device of claim 32 also comprising:
a) the support member having a perimeter area;
b) the adhesive and the composite membrane being located on the support member within a placement area defined by the perimeter area; and
c) a pad barrier being connected to the support member about the perimeter area.

34. The biomolecule binding device of claim 30 wherein the support member is selected from the group consisting of glass or a plastic polymer.

35. A multiwell biomolecule binding device comprising
a) a multiwell plate having a well bottom support surface and a plurality of well, each such well having a well opening and being separate and distinct from the other wells; and
b) a plurality of well membranes, each well membrane being comprised of a track etched porous surface having an average pore size of about 40 um or less, a plurality of interstitial surfaces forming the pores of the track etched porous surface, and a microporous, biomolecule binding polymer dispersed about at least the interstitial surfaces; each well having a well membrane placed therein.

36. The multiwell biomolecule device of claim 33 also comprising an adhesive being located on at least a portion of the well bottom support in each well and between each well membrane and the well bottom support, thereby allowing for each well membrane to adhere to the well bottom support surface.

37. A process for making a biomolecule binding device comprising:
a) selecting a support member;
b) placing a dry film adhesive onto the support member;
c) placing on the dry film, heat-activated adhesive a composite membrane comprised of a track etched porous surface having an average pore size of about 40 um or less, a plurality of interstitial surfaces forming the pores of the track etched porous surface, and a microporous, biomolecule binding polymer dispersed about at least the interstitial surfaces; and
d) applying thermal energy to the adhesive sufficient to cause the adhesive to adhere the composite membrane to the support member.

38. The process of claim 37 wherein the track etched porous surface is selected from the group consisting of polyimide polymers, fluorinated polymers, polyethylene naphthalate polymers, polypropylene polymers, polycarbonate polymers, polyester polymers, or polycarbonate polymers.

39. The process of claim 37 also comprising;
a) the support member having a perimeter area, the adhesive and the composite membrane being placed on the support member within a placement area defined by the perimeter area; and
b) a pad barrier also being connected to the support about the perimeter area.

40. The process of claim 37 wherein the support member is selected from the group consisting of glass or a plastic polymer.

41. A process for making a multiwell biomolecule binding device comprising
a) selecting a multiwell plate having a well bottom support surface and a plurality of well, each such well having a well opening and being separate and distinct from the other wells;
b) placing over the well openings a composite membrane comprised of a track etched porous surface having an average pore size of about 40 um or less, a plurality of interstitial surfaces forming the pores of the track etched porous surface, and a microporous, biomolecule binding polymer dispersed about at least the interstitial surfaces; and
c) cutting a plurality of well membranes from the composite membrane, a well membrane being cut so as to be placed into a single well.

42. The process of claim 42 wherein an adhesive is placed on at least a portion of the well bottom support in each well before the plurality of well membranes are cut, thereby allowing for each well membrane to adhere to the well bottom support surface.

43. A process for detecting an analyte signal from a biomolecule binding assay having a assay signal generation reagent comprising:
a) selecting a biomolecule binding device comprised of a support member having a support surface; and a composite membrane attached to the support surface, the composite membrane being comprised of a track etched porous surface having an average pore size of about 40 um or less, a plurality of interstitial surfaces forming the pores of the track etched porous surface, and a microporous, biomolecule binding polymer dispersed about at least the interstitial surfaces;
b) placing an analyte sample on the composite membrane, thereby allowing any biomolecule analyte in the sample to bind to the composite membrane;
c) reacting the assay signal generation reagent with the bound biomolecule so as to form a labelled and bound biomolecule; and
d) detecting the labelled and bound biomolecule.

44. The process of claim 43 wherein the track etched porous surface is selected from the group consisting of polyimide polymers, fluorinated polymers, polyethylene naphthalate polymers, polypropylene polymers, polycarbonate polymers, polyester polymers, or polycarbonate polymers.

45. The process of claim 43 wherein the biomolecule binding assay is selected from the group consisting of immunoassays, protein microarrays, nucleic acid assays, receptor binding assays, DNA protein binding reactions, or immobilize enzyme reaction assays.

46. A readable biomolecule binding assay device for detecting an analyte signal from a biomolecule binding assay having a assay signal generation reagent comprising:
a.) a biomolecule binding device comprised of a support member having a support surface; and a composite membrane attached to the support surface, the composite membrane being comprised of a track etched porous surface having an average pore size of less than 40 um, a plurality of interstitial surfaces forming the pores of the track etched porous surface, and a microporous, biomolecule binding polymer dispersed about at least the interstitial surfaces;
b.) an analyte sample disposed on the composite membrane, thereby allowing any biomolecule analyte in the sample to be bound to the composite membrane; and
c.) the assay signal generation reagent reacted with any bound biomolecule so as to form a labelled and bound biomolecule.

47. The readable biomolecule binding assay device of claim 46 wherein the track etched porous surface is selected from the group consisting of polyimide polymers, fluorinated polymers, polyethylene naphthalate polymers, polypropylene polymers, polycarbonate polymers, polyester polymers, or polycarbonate polymers.

48. The device of claim 46 wherein the biomolecule binding assay is selected from the group consisting of immunoassays or protein microarrays.
